(19) United States
(12) Patent Application Publication
(10) Pub. No.: US 2003/0232343 A1
(43) Pub. Date: Dec. 18, 2003

(54) Methods for testing reagent distribution in reaction chambers

(76) Inventors: Eric M. Leproust, Campbell, CA (US); Douglas A. Amorese, Los Altos, CA (US); Bill J. Peck, Mountain View, CA (US)

Correspondence Address: AGILENT TECHNOLOGIES, INC., Legal Department, DL429, Intellectual Property Administration, P.O. Box 7599, Loveland, CO, 80537-0599, US

(21) Appl. No.: 10/172,675

(22) Filed: Jun. 14, 2002

(51) Int. Cl. C12Q 1/68; G01N 33/53; G01N 33/543

(52) U.S. Cl. 435/6; 435/7.1; 436/518

(57) ABSTRACT

Apparatus and methods are disclosed for determining a functional property of a fluid in a chamber. A support to which is bound a plurality of test elements is introduced into the chamber. Each of the test elements comprises a reaction domain and a detection domain. A fluid that is interactive with the reaction domains is introduced into the chamber. Fluid is removed from the chamber. The locations at which the fluid has not interacted with the reaction domains is determined by means of the detection domains. The locations are then related to the functional property of the fluid.

### BACKGROUND OF THE INVENTION

This invention relates to chemical reactions performed in reaction chambers where fluid reagents are flowed into and out of the reaction chamber as a part of the chemical reactions. In one aspect the invention relates to the manufacturing of supports having bound to the surfaces thereof a plurality of chemical compounds such as polymers, which are prepared on the surface in a series of steps. More particularly, the present invention relates to methods for solid phase chemical synthesis, particularly solid phase synthesis of oligomer arrays, or attachment of oligonucleotides and polynucleotides to surfaces, e.g., arrays of polynucleotides.

In the field of diagnostics and therapeutics, it is often useful to attach species to a surface. One important application is in solid phase chemical synthesis wherein initial derivatization of a substrate surface enables synthesis of polymers such as oligonucleotides and peptides on the substrate itself. Support bound oligomer arrays, particularly oligonucleotide arrays, may be used in screening studies for determination of binding affinity. Modification of surfaces for use in chemical synthesis has been described. See, for example, U.S. Pat. No. 5,624,711 (Sundberg), U.S. Pat. No. 5,266,222 (Willis) and U.S. Pat. No. 5,137,765 (Farnsworth).

Determining the nucleotide sequences and expression levels of nucleic acids (DNA and RNA) is critical to understanding the function and control of genes and their relationship, for example, to disease discovery and disease management. Analysis of genetic information plays a crucial role in biological experimentation. This has become especially true with regard to studies directed at understanding the fundamental genetic and environmental factors associated with disease and the effects of potential therapeutic agents on the cell. Such a determination permits the early detection of infectious organisms such as bacteria, viruses, etc.; genetic diseases such as sickle cell anemia; and various cancers. This paradigm shift has lead to an increasing need within the life science industries for more sensitive, more accurate and higher-throughput technologies for performing analysis on genetic material obtained from a variety of biological sources.

Unique or misexpressed nucleotide sequences in a polynucleotide can be detected by hybridization with a nucleotide multimer, or oligonucleotide, probe. Hybridization is based on complementary base pairing. When complementary single stranded nucleic acids are incubated together, the complementary base sequences pair to form double stranded hybrid molecules. These techniques rely upon the inherent ability of nucleic acids to form duplexes via hydrogen bonding according to Watson-Crick base-pairing rules. The ability of single stranded deoxyribonucleic acid (ssDNA) or ribonucleic acid (RNA) to form a hydrogen-bonded structure with a complementary nucleic acid sequence has been employed as an analytical tool in molecular biology research. An oligonucleotide probe employed in the detection is selected with a nucleotide sequence complementary, usually exactly complementary, to the nucleotide sequence in the target nucleic acid. Following hybridization of the probe with the target nucleic acid, any oligonucleotide probe/nucleic acid hybrids that have formed are typically separated from unhybridized probe. The amount of oligonucleotide probe in either of the two separated media is then tested to provide a qualitative or quantitative measurement of the amount of target nucleic acid originally present.

Direct detection of labeled target nucleic acid hybridized to surface-bound polynucleotide probes is particularly advantageous if the surface contains a mosaic of different probes that are individually localized to discrete, known areas of the surface. Such ordered arrays containing a large number of oligonucleotide probes have been developed as tools for high throughput analyses of genotype and gene expression. Oligonucleotides synthesized on a solid support recognize uniquely complementary nucleic acids by hybridization, and arrays can be designed to define specific target sequences, analyze gene expression patterns or identify specific allelic variations. The arrays may be used for conducting cell study, for diagnosing disease, identifying gene expression, monitoring drug response, determination of viral load, identifying genetic polymorphisms, analyze gene expression patterns or identify specific allelic variations, and the like.

In one approach, cell matter is lysed, to release its DNA as fragments, which are then separated out by electrophoresis or other means, and then tagged with a fluorescent or other label. The resulting DNA mix is exposed to an array of oligonucleotide probes, whereupon selective binding to matching probe sites takes place. The array is then washed and interrogated to determine the extent of hybridization reactions. In one approach the array is imaged so as to reveal for analysis and interpretation the sites where binding has occurred. Arrays of different chemical probe species provide methods of highly parallel detection, and hence improved speed and efficiency, in assays. Assuming that the different sequence polynucleotides were correctly deposited in accordance with the predetermined configuration, then the observed binding pattern will be indicative of the presence and/or concentration of one or more polynucleotide components of the sample.

Biopolymer arrays can be fabricated using either in situ synthesis methods or deposition of previously obtained biopolymers. In general, arrays are synthesized on a surface of a substrate by one of any number of synthetic techniques that are known in the art. The in situ and deposition techniques are often carried out in a reaction chamber where reagents are applied to the surface of a support. Application of the reagents depends on the nature of the technique. In one approach, photolithographic methods are employed. In another approach reagents are applied as droplets to the surface of a support. In the aforementioned techniques there are usually one or more steps of the reaction scheme that are performed by placing the support into a chamber and introducing a fluid reagent into the chamber, which results in flooding of the surface of the support with the fluid reagent. For example, the reaction scheme may involve one or more steps such as reacting the surface with the desired reagent to form the chemical compound, washing the surface, oxidizing the compounds present on the surface, deblocking sites on the compounds present on the surface, and so forth. In one approach to the synthesis of microarrays flow cells or flow devices are employed in which a substrate is placed to carry out parts of the synthesis procedure.

In methods involving reaction chambers where fluid reagents are flowed into and out of the chamber, there is concern over the flow pattern and distribution of the fluid reagent within the reaction chamber. As may be appreciated, there may be some areas within the reaction chamber where the distribution of fluid reagent is significantly different than in other areas. Accordingly, the spatial uniformity of the chemical reactions being performed may be comprised. For example, where multiple chemical compounds are synthesized on the surface of a support at predetermined sites, lack of spatial uniformity results in some sites not having the desired chemical compound because a fluid reagent was not properly distributed to all sites on the surface of the support.

There is a need, therefore, for a method for determining functional properties of liquid reagents in flow devices. The functional properties include, e.g., the flow characteristics and reagent distribution of liquid reagents in flow devices. The method should provide knowledge of functional properties to provide for spatial uniformity of chemical reactions being performed on the surface of supports within the flow devices.

### SUMMARY OF THE INVENTION

One embodiment of the present invention is a method for determining a functional property of a fluid in a chamber. A support to which is bound a plurality of test elements is introduced into the chamber. Each of the test elements comprises a reaction domain and a detection domain. A fluid that is interactive with the reaction domains is introduced into the chamber. Fluid is removed from the chamber. The locations at which the fluid has not interacted with the reaction domains are determined by means of the detection domains. The locations are then related to the functional property of the fluid.

Another embodiment of the present invention is a method for determining the flow pattern or reagent distribution of a liquid reagent in a chamber. A support to which is bound a plurality of test elements is introduced into the chamber. Each of the test elements comprises a first portion proximal the support and a second portion distal the support. The first portion comprises (N)ₙ wherein N is a nucleotide and n is about 5 to about 50. The second portion comprises a polynucleotide. A liquid reagent that is reactive with the reaction domains is introduced into the chamber. The liquid reagent is removed from the chamber and the support is exposed to a cleavage reagent that cleaves only those first portions that have reacted with the liquid reagent. The support is exposed to a complementary polynucleotide comprising a detectable label. The support is examined for the locations at which the detectable label is present. The locations are related to the flow pattern and/or the reagent distribution of the liquid reagent.

Another embodiment of the present invention is a method for synthesizing an array of biopolymers on the surface of a support wherein the synthesis comprises a plurality of monomer additions. After each of the monomer additions the support is placed into a chamber and subjected to the method described above and subsequently subjected to at least one wash step. The surface of the support in the chamber is subjected to a step of the synthesis that is subsequent to a monomer addition.

Another embodiment of the present invention is a method for synthesizing an array of biopolymers on the surface of a support. The support is placed into a reaction chamber. The biopolymers or precursors of the biopolymers are applied to the surface of the support. The support is removed from the reaction chamber and placed into a flow chamber. The flow chamber has previously been subjected to a method as described above and to at least one wash step. A liquid reagent for carrying out the synthesis of the biopolymers is introduced into the flow chamber. The flow parameters of the flowing liquid are adjusted based on the results of the determination of the aforementioned method. The support is removed from the flow chamber and the above steps are repeated to form the array of biopolymers.

Another embodiment of the present invention is a support comprising a plurality of features on a surface of the support. The features comprise a first portion proximal the surface and a second portion distal the surface. The first portion comprises (N)ₙ wherein N is a nucleotide and n is about 5 to about 50, and the second portion comprises a polynucleotide.

Another embodiment of the present invention is a method for correcting for flow irregularities of a fluid in a chamber. A support to which is bound a plurality of test elements is introduced into the chamber. Each of the test elements comprises a reaction domain and a detection domain. Then, a fluid that is interactive with the reaction domains is introduced into the chamber and subsequently removed from the chamber. The locations at which the fluid has not interacted with the reaction domains are determined by means of the detection domains. The locations are related to the functional property of the fluid. One or more fluid flow parameters of the chamber are adjusted based on the above determination.

Another embodiment of the invention is a kit comprising in packaged combination (a) a support to which is bound a plurality of test elements, each of the test elements comprising a reaction domain and a detection domain, (b) one or more reagent solutions comprising reagents reactive with the reaction domain, and (c) optionally, reagents binding with the detection domain.

### BRIEF DESCRIPTION OF THE DRAWINGS

FIG. 1 is a schematic diagram depicting one aspect of the present invention.

FIG. 2 is an alternate diagram depicting one aspect of the present invention.

FIG. 3 is an alternate diagram depicting one aspect of the present invention.

FIG. 4A is a schematic diagram depicting a depurination reaction.

FIG. 4B is a schematic diagram depicting a base-induced cleavage of a phosphate backbone.

FIG. 5 is a three-dimensional depiction of a spatial distribution of a hybridization signal on an array.

FIG. 6 is a two-dimensional depiction of a spatial distribution of a hybridization signal on an array.

### DETAILED DESCRIPTION OF THE INVENTION

The present invention utilizes a plurality of multi-domain test elements that is arranged on the surface of a support. Usually, two domains are employed for the test elements and one of the domains of the test elements is attached to the surface of the support and the other of the domains is attached to the first domain. The domains are chosen so that the domain proximal the surface of the support (arbitrarily referred to herein as the first domain) can undergo an alteration so that, upon further treatment, altered domains may be identified and distinguished from unaltered domains. In one approach, the domain proximal the surface of the support is reactive with a liquid reagent and the domain distal the support (arbitrarily referred to herein as the second domain) provides a detection moiety. The support with the test elements is placed into the chamber of a flow device and the support is exposed to a liquid reagent that is reactive with the first domain. The alteration of the domain proximal the support renders the domain susceptible to cleavage. Upon cleavage of the altered domains, the cleaved product comprises part of the altered domain and also the second domain that is distal the support. The non-cleaved domains are the unaltered domains, which would retain the second domain. The support is then exposed to a labeled reagent that is interactive with the second domain. Usually, the labeled reagent interacts with the second domain by specific binding. When the label is subjected to signal determination, the locations of intact test elements may be identified. The signal intensity is inversely proportional to the extent of the reaction between the reactive domain and the liquid reagent. Accordingly, the spatial distribution of the signal can be correlated with the original distribution of reactive agent in the liquid reagent and/or with the original flow pattern of reagent in the chamber of the flow device.

The number of test elements on the surface of the support is usually chosen to correspond substantially to the number of features on the surface of a support in a particular chemical reaction that is carried out on the surface of the support. In this way a reliable and usable determination of the flow properties of a liquid in the chamber may be obtained. The number of elements on the surface is about 0.1 to about 10⁴ per mm², usually, about 100 to about 2000 per mm².

The first domain may be selected from a number of different moieties. One requirement of the first domain is that it is attachable to the surface of a support in a substantially irreversible manner under the conditions to which the support is exposed. By the term “substantially irreversible” is meant that the first domain cannot be severed from the support to an extent that would impact the results obtained in the present methods. Preferably, the first domain is irreversibly attached to the support under the conditions to which the support is exposed.

Another requirement for the first domain is that it is able to undergo an alteration by reaction with the liquid reagent such that the altered first domain comprises a releasable fragment that comprises part of the first domain and the second domain. It is desirable in the present invention that the alteration reaction that occurs in the presence of the reactive liquid be a relatively slow reaction. A basic premise in the present invention is to determine functional properties of a liquid reagent introduced into a flow device. It is important in the present invention that alteration reaction not be so robust as to alter a substantial part or all of the molecules of the first domain that are present on the surface of a support. In general, the first domain and the reactive liquid reagent are chosen so that under the conditions of the reaction about 1 to about 20%, usually, about 5 to about 10%, of the molecules of the first domain are altered in a period of about 0.1 to about 20 minutes, usually, about 1 to about 10 minutes.

With the above considerations in mind, the first domain may be any molecule that comprises a functionality that is directly cleavable by exposure to a reactive liquid reagent or is capable of being rendered cleavable as a result of an alteration resulting from exposure to a reactive liquid reagent. The releasable functionality may be, for example, a cleavable functionality, i.e., a functionality that is subject to cleavage by another reagent. The cleavable functionality may be a bond or a linking functionality. The following are examples of suitable cleavable moieties, by way of illustration and not limitation and with the proviso that the above reactivity parameters apply: base-cleavable sites such as esters, particularly succinates (as mentioned above) (cleavable by, e.g., ammonia or trimethylamine), quaternary ammonium salts (cleavable by, e.g., aqueous sodium hydroxide), acid-cleavable sites such as, e.g., benzyl alcohol derivatives (cleavable using trifluoroacetic acid), teicoplanin aglycone (cleavable by trifluoroacetic acid followed by base), acetals and thioacetals (cleavable by trifluoroacetic acid), thioethers (cleavable, e.g., by HF or cresol) and sulfonyls (cleavable by trifluoromethane sulfonic acid, trifluoroacetic acid, thioanisole, or the like); nucleophile-cleavable sites such as phthalamide (cleavable with substituted hydrazines), ester (cleavable with, e.g., aluminum trichloride) and Weinreb amide (cleavable with lithium aluminum hydride) and other types of chemically cleavable sites, including phosphorothioate (cleavable with silver or mercuric ions) and diisopropyldialkoxysilyl (cleavable with fluoride ion) and electron-rich olefins such as enol ethers, enamines and vinyl sulfides, and heterocycles such as thiazole and oxazole. Other cleavable sites will be apparent to one skilled in the art such as those disclosed in, for example, Brown, Contemporary Organic Synthesis (1997) 4(3):216-237.

The releasable fragment may be released from the surface of the support directly by action of the reactive liquid or it may be released indirectly by subsequent treatment with a cleavage agent. Usually, the altered first domain provides a released fragment and an attached fragment, i.e., a fragment of the first domain that remains attached to the surface of the support and that no longer comprises the second domain. The nature of the attached fragment depends on the nature of the alteration to the first domain and, ultimately, on where a releasable functionality is introduced into the first domain. Another requirement for the first domain is that the remains of the first domain attached to the surface of the support do not interact with the target complementary to the second domain.

The first domain may be a synthetic material or a material derived from a natural source. The first domain may comprise a polymer such as an addition or condensation polymer. The polymer can be comprised of polystyrene, polyacrylamide, homopolymers and copolymers of derivatives of acrylate and methacrylate, particularly esters and amides, silicones and the like. The first domain may be a homooligomer or a heterooligomer having different monomers of the same or different chemical characteristics, e.g., nucleotides and amino acids. In accordance with the present invention, the polymer comprises a bond or a functional moiety that may be altered and subsequently cleaved or that may be cleaved directly. As may be evident from the discussion herein, the nature of the cleavable bond or linkage determines the nature of the reactive liquid reagent that is employed.

In one embodiment the first domain comprises an oligomer of nucleotides, which may be the same or different. The oligomer may comprise about 2 to about 200 nucleotides, usually, about 5 to about 40 nucleotides. The oligomer may be an oligonucleotide represented by the formula (N)ₙ wherein N is a nucleotide and n is 2 to about 200. In a preferred embodiment all nucleotides are the same and are selected from the group consisting of A, T, G, C and U wherein the letter abbreviations refer to the base of the nucleotide, namely, adenine, thymine, guanine, cytosine and uracil, respectively. In this embodiment the liquid reagent may be one that results in altering the oligonucleotide by facilitating a depurination or de-pyrimidation reaction. Such liquid reagents generally comprise an acid such as, for example, a carboxylic acid, which may be substituted or unsubstituted. The carboxylic acid may comprise about 1 to about 30 carbon atoms, preferably, about 2 to about 10 carbon atoms. The carboxylic acids include, for example, acetic acid, propionic acid, butyric acid, pentanoic acid, hexanoic acid and so forth. The carboxylic acids may be saturated or unsaturated, substituted or unsubstituted. Substituents that may be present on the carboxylic acid include, by way of illustration and not limitation, fluorine, chlorine, bromine, iodine, nitro, substituted or unsubstituted benzoic acids, and the like. For depurination or depyrimidation the carboxylic acid should have a pKa of about 0 to about 5, preferably, about 2 to about 3. As a specific example, the first domain comprises polyA or (A)n wherein n is as defined above. The carboxylic acid liquid reagent may be, for example, acetic acid or substituted acetic acid such as mono-, di-, or trichloroacetic acid.

The support to which a plurality of chemical compounds is attached is usually a porous or non-porous water insoluble material. The support can have any one of a number of shapes, such as strip, plate, disk, rod, particle, and the like. The support can be hydrophilic or capable of being rendered hydrophilic or it may be hydrophobic. The support is usually glass such as flat glass whose surface has been chemically activated to support binding or synthesis thereon, glass available as Bioglass and the like. However, the support may be made from materials such as inorganic powders, e.g., silica, magnesium sulfate, and alumina; natural polymeric materials, particularly cellulosic materials and materials derived from cellulose, such as fiber containing papers, e.g., filter paper, chromatographic paper, etc.; synthetic or modified naturally occurring polymers, such as nitrocellulose, cellulose acetate, poly (vinyl chloride), polyacrylamide, cross linked dextran, agarose, polyacrylate, polyethylene, polypropylene, poly(4-methylbutene), polystyrene, polymethacrylate, poly(ethylene terephthalate), nylon, poly(vinyl butyrate), etc.; either used by themselves or in conjunction with other materials; ceramics, metals, and the like. Preferably, for packaged arrays the support is a non-porous material such as glass, plastic, metal and the like.

The first domain is attached to the surface of the support employing methods that are known in the art. Depending on the nature of the first domain such as, e.g., where the first domain is an oligonucleotide, the first domain may be synthesized in situ on the surface of the support. Alternatively, preformed first domain molecules may be attached at one of their ends to the surface of the support. The surface of a support is normally treated to create a primed or functionalized surface, that is, a surface that is able to react with either monomeric units that form the first domain or with the preformed first domain molecule. Functionalization relates to modification of the surface of a support to provide a plurality of functional groups on the support surface. By the term “functionalized surface” is meant a support surface that has been modified so that a plurality of functional groups are present thereon. The manner of treatment is dependent on the nature of the first domain and on the nature of the support surface. In one approach a reactive hydrophilic site or reactive hydrophilic group is introduced onto the surface of the support. Such hydrophilic moieties can be used as the starting point in a synthetic organic process.

In one embodiment, the surface of the support, such as a glass support, is siliceous, i.e., comprises silicon oxide groups, either present in the natural state, e.g., glass, silica, silicon with an oxide layer, etc., or introduced by techniques well known in the art. One technique for introducing siloxyl groups onto the surface involves reactive hydrophilic moieties on the surface. These moieties are typically epoxide groups, carboxyl groups, thiol groups, and/or substituted or unsubstituted amino groups as well as a functionality that may be used to introduce such a group such as, for example, an olefin that may be converted to a hydroxyl group by means well known in the art. One approach is disclosed in U.S. Pat. No. 5,474,796 (Brennan), the relevant portions of which are incorporated herein by reference. A siliceous surface may be used to form silyl linkages, i.e., linkages that involve silicon atoms. Usually, the silyl linkage involves a silicon-oxygen bond, a silicon-halogen bond, a silicon-nitrogen bond, or a silicon-carbon bond.

Another method for attachment is described in U.S. Pat. No. 6,219,674 (Fulcrand, et al.). A surface is employed that comprises a linking group consisting of a first portion comprising a hydrocarbon chain, optionally substituted, and a second portion comprising an alkylene oxide or an alkylene imine wherein the alkylene is optionally substituted. One end of the first portion is attached to the surface and one end of the second portion is attached to the other end of the first portion chain by means of an amine or an oxy functionality. The second portion terminates in an amine or a hydroxy functionality. The surface is reacted with the substance to be immobilized under conditions for attachment of the substance to the surface by means of the linking group.

Another method for attachment is described in U.S. Pat. No. 6,258,454 (Lefkowitz, et al.). A solid support having hydrophilic moieties on its surface is treated with a derivatizing composition containing a mixture of silanes. A first silane provides the desired reduction in surface energy, while the second silane enables functionalization with molecular moieties of interest, such as small molecules, initial monomers to be used in the solid phase synthesis of oligomers, or intact oligomers. Molecular moieties of interest may be attached through cleavable sites.

A procedure for the derivatization of a metal oxide surface uses an aminoalkyl silane derivative, e.g., trialkoxy 3-aminopropylsilane such as aminopropyltriethoxy silane (APS), 4-aminobutyltrimethoxysilane, 4-aminobutyltriethoxysilane, 2-aminoethyltriethoxysilane, and the like. APS reacts readily with the oxide and/or siloxyl groups on metal and silicon surfaces. APS provides primary amine groups that may be used to carry out the present methods. Such a derivatization procedure is described in EP 0 173 356 B1, the relevant portions of which are incorporated herein by reference. Other methods for treating the surface of a support will be suggested to those skilled in the art in view of the teaching herein.

The second domain is usually a member of a specific binding pair. A member of a specific binding pair is one of two different molecules, having an area on the surface or in a cavity that specifically binds to and is thereby defined as complementary with a particular spatial and polar organization of the other molecule. The members of the specific binding pair are sometimes referred to as ligand and receptor (antiligand). The members may be, for example, members of an immunological pair such as antigen-antibody and the like, biotin-avidin, hormones-hormone receptors, nucleic acid duplexes, IgG-protein A, polynucleotide pairs such as DNA-DNA, DNA-RNA, protein-nucleic acid complexes, and the like. In a preferred embodiment, especially where the first domain comprises a homooligomer of nucleotides, the second domain is a heterooligomer of nucleotides. In the latter situation the second domain is preferably DNA or RNA.

The second domain or detection domain is substantially irreversibly attached to the first domain and is stable under the conditions employed. The first domain and the second domain may be attached prior to attachment of the first domain to the surface of the support. Alternatively, the second domain may be attached to the first domain after the latter has been attached to the support. Where the first and second domains comprise nucleotides, the second domain may be synthesized in situ after the synthesis of the first domain on the surface of the support.

The linkage between the first domain and the second domain is generally stable to the action of the reactive liquid reagent so that the second domain remains attached to the released fragment of the first domain upon release thereof from the first domain. Attachment of the first domain and the second domain may be direct (such as by a bond between the two domains) or indirect (such as by a linking group between the two domains), covalent or non-covalent and can be accomplished by well-known techniques, commonly available in the literature. See, for example, “Immobilized Enzymes,” Ichiro Chibata, Halsted Press, New York (1978) and Cuatrecasas, J. Biol. Chem., 245:3059 (1970). A wide variety of functional groups are available or can be incorporated. Functional groups include carboxylic acids, aldehydes, amino groups, cyano groups, ethylene groups, hydroxyl groups, mercapto groups, and the like. The manner of linking a wide variety of compounds is well known and is amply illustrated in the literature (see above). The length of a linking group between the first domain and the second domain may vary widely, depending upon the nature of the first and second domains, the effect of the distance on the specific binding properties and the like.

In one embodiment of the present invention the second domain may comprise a detectable label, which is a chemical entity capable of being detected directly or indirectly by a suitable detection means. Labels include, for example, luminescent molecules such as fluorescers, chemiluminescers, and the like, enzymes, coenzymes, radiolabels, and so forth. Suitable enzymes and coenzymes are disclosed in Litman, et al., U.S. Pat. No. 4,275,149, columns 19-28, and Boguslaski, et al., U.S. Pat. No. 4,318,980, columns 10-14; suitable fluorescers and chemiluminescers are disclosed in Litman, et al., U.S. Pat. No. 4,275,149, at columns 30 and 31; which are incorporated herein by reference.

The label may be conjugated to the second domain by procedures well known in the art. The conjugation should be irreversible under the conditions employed in the present methods. Typically, the label contains a functional group suitable for attachment to the second domain. The functional groups suitable for attaching the label are usually activated esters or alkylating agents. Details of techniques for attaching labels are well known in the art. See, for example, Matthews, et al., Anal. Biochem. (1985) 151:205-209 and Engelhardt, et al., European Patent Application No. 0302175.

The label is usually a member of a signal producing system, which may have one or more components, at least one of which is the label. The signal producing system includes all of the reagents required to produce a measurable signal. Other components of the signal producing system can include substrates, coenzymes, enhancers, activators, chemiluminescent compounds, cofactors, inhibitors, scavengers, specific binding substances, and the like. If the label is an enzyme, additional members of the signal producing system include enzyme substrates and so forth.

The detection means depend on the nature of the label. If the label is a fluorescent molecule, the medium can be irradiated and the fluorescence determined. If the label is an enzyme, the product of the enzyme reaction is preferably a luminescent product, or a fluorescent or non-fluorescent dye, any of which can be detected spectrophotometrically, or a product that can be detected by other spectrometric or electrometric means. Where the label is a radioactive group, the medium can be counted to determine the radioactive count.

In some embodiments the second domain is not labeled and a detection reagent is added to the support subsequent to the cleavage reaction. In this approach, it is usually necessary to carry out a separation step wherein cleaved moieties that comprise the second domain are separated from the support. Such a separation step generally involves washing the surface of the support to remove unbound materials. The detection reagent usually comprises a member of the specific binding pair that is complementary to the second domain and further comprises a detectable label. The nature of the label is discussed above. The attachment of the label to the complementary member of the specific binding pair may be achieved in the same manner as that described above for attachment of a label to the second domain.

It is also within the purview of the present invention to carry out the signal determination in the absence of a separation step. In this situation both a labeled second domain and a label reagent are employed. The labels are related in that they interact with one another only when brought into proximity by the binding of the label reagent to the labeled second domain. In this approach the label pair may be a fluorescer-quencher pair, an enzyme pair where the product of one enzyme is the substrate for the other enzyme, a chemiluminescent compound label that is activated by singlet oxygen generated by irradiation of a photosensitizer label, and so forth. The interaction between such labels is sometimes referred to as a “channeling” reaction.

The aforementioned support comprising the first and second domains as test elements may be employed in a method for determining a functional property of a fluid in a chamber. The fluid may be a gas or a liquid reagent. A support to which is bound a plurality of test elements is introduced into a chamber of a flow device. A fluid that is interactive with the reaction domains is introduced into the chamber. Typically, the flow cell is a housing having a reaction cavity or chamber disposed therein. The flow cell allows fluids to be introduced into the chamber and removed from the chamber where the support is disposed. The support is mounted in the chamber in or on a holder. The housing usually further comprises at least one fluid inlet and at least one fluid outlet for flowing fluids into and through or out of the chamber in which the support is mounted.

The housing of the flow cell is generally constructed to permit access into the chamber therein. In one approach, the flow cell has an opening that is sealable to fluid transfer after the support is placed therein. Such seals may comprise a flexible material that is sufficiently flexible or compressible to form a fluid tight seal that can be maintained under increased pressures encountered in the use of the device. The flexible member may be, for example, rubber, flexible plastic, flexible resins, and the like and combinations thereof. In any event the flexible material should be substantially inert with respect to the fluids introduced into the device and must not interfere with the reactions that occur within the device. The flexible member is usually a gasket and may be in any shape such as, for example, circular, oval, rectangular, and the like. Preferably, the flexible member is in the form of an O-ring.

In another approach the housing of the flow cell may be conveniently constructed in two parts, which may be referred to generally as top and bottom elements. These two elements are sealably engaged during synthetic steps and are separable at other times to permit the support to be placed into and removed from the chamber of the flow cell. Generally, the top element is adapted to be moved with respect to the bottom element although other situations are contemplated herein. Movement of the top element with respect to the bottom element is achieved by means of, for example, pistons, and so forth. The movement is controlled electronically by means that are conventional in the art. In another approach a reagent chamber is formed in situ from a support and a sealing member.

The inlet of the flow cell is usually in fluid communication with an element that controls the flow of fluid into the flow cell such as, for example, a manifold, a valve, and the like or combinations thereof. This element in turn is in fluid communication with a dispensing station containing the desired fluid reagent. Any reagent that is normally a solid reagent may be converted to a fluid reagent by dissolution in a suitable solvent, which may be a protic solvent or an aprotic solvent. The nature of the solvent is determined by the nature of the reagent that is reactive with the first domain. Accordingly, the solvent may be an organic solvent such as, by way of illustration and not limitation, oxygenated organic solvents of from 1 to about 6, more usually from 1 to about 4, carbon atoms, including alcohols such as methanol, ethanol, propanol, etc., ethers such as tetrahydrofuran, ethyl ether, propyl ether, etc., acetonitrile, dimethylformamide, dimethylsulfoxide, dichloromethane, toluene, and the like. The solvent may be an aqueous medium that is solely water or may contain a buffer, or may contain from about 0.01 to about 80 or more volume percent of a cosolvent such as an organic solvent as mentioned above.

In one embodiment the fluid dispensing stations are affixed to a base plate or main platform to which the flow cells are mounted. Any fluid dispensing station may be employed that dispenses fluids such as water, aqueous media, organic solvents and the like. The fluid dispensing station may comprises a pump for moving fluid and may also comprise a valve assembly and a manifold as well as a means for delivering predetermined quantities of fluid to the flow cell. The fluids may be dispensed by pumping from the dispensing station. In this regard any standard pumping technique for pumping fluids may be employed in the present apparatus. For example, pumping may be by means of a peristaltic pump, a pressurized fluid bed, a positive displacement pump, e.g., a syringe pump, and the like.

After the reactive liquid reagent in introduced into the flow cell, the reagent is held in contact with the support for a time and under conditions sufficient for a proportional number of the first domains to become altered depending on the flow properties of the liquid reagent and/or the reagent distribution in the liquid reagent. The relationship between the time periods and the extent of alteration of the first domains is discussed above. In general, the time periods and conditions are dependent on the nature of the reactive reagent and the nature of the first domain.

Fluid is then removed from the chamber by gravity, suction, vacuum, introduction of pressurized gas and so forth. If the reactive fluid reagent brings about the cleavage of the first domain, there is no need to add any additional reagents and the support may be treated to remove unbound reagents as described hereinbelow. If the reactive fluid reagent alters, but does not cleave, the first domain, then a reagent must be added to induce cleavage in the altered region of the first domain. The nature of the reagent depends on the nature of the alteration. The depurination of a polyA first domain by a carboxylic acid is an example of an alteration that requires further treatment (FIG. 4A). In the polyA example given above, the depurinated material is subjected to treatment with an appropriate basic solution to bring about cleavage in the first domain (FIG. 4B). In general, the basic solution must have sufficient basicity to bring about cleavage of the depurinated first domain. The base should have a conjugate acid of pKa of about 9 to 12 and should not cleave the attachment linkage between the DNA oligonucleotide and the glass surface. The basic solution may be, for example, ethanolamine, mixtures of an alkyl diamine such as, e.g., ethylene diamine, methyl amine, etc., with a lower alkyl alcohol such as, e.g., ethanol, and the like. Specific examples, by way of illustration and not limitation, include 1:1 ethylene diamine:ethanol, 1:1 methyl armine:ethanol, and so forth. Other cleavage reagents will be apparent to one skilled in the art in view of the disclosure hereinabove.

Next, the support is treated to remove unbound reagents from its surface. To this end the support may be subjected to one or more wash steps, which conveniently can be carried out in the flow chamber. On the other hand, the support may be removed from the flow chamber and washed in a different location such as a wash station. The wash reagent may, but need not, be the solvent for the fluid reagent mentioned above. The primary concern in washing the surface of the support is to remove unbound materials that might interfere in the step of examining the surface of the support for the presence and location of signal from the label.

When the support has been separated from unbound materials, the surface of the support is examined for the presence and location of signal. The manner in which the signal determination is made is dependent on the nature of the label as discussed above. If the second domain is labeled, the surface of the support is examined directly for the presence and location of signal. If the second domain is not labeled, a labeled reagent is added to the surface of the support. The labeled reagent binds specifically at locations where the second domain is present. The locations at which the fluid has not interacted with the reaction domains are determined by means of the detection domains. The locations are then related to the functional property of the fluid.

For example, the flow characteristics and/or reagent distribution of the fluid reagent in the flow chamber may be determined by observing the signal from the surface of the support. The presence of signal at certain locations on the surface indicates that the test element comprising the first and second domains is still intact. This means that the flow of reagent to the areas where the signal is found was not as great as the flow to areas where no signal was found. As is evident from the above discussion, where the flow of reagent occurs, there is a greater opportunity for the reactive fluid reagent to react with the first domain. When the first domain is altered, it is either cleaved directly or rendered cleavable by such alteration. Cleavage of the first domain ultimately results in the absence of signal at the location of such cleavage. Thus, the signal intensity is inversely proportional to the extent of the reaction between the first domain or the reactive domain and the reagent present in the fluid reagent.

The manner of observing the signal depends on the nature of the label. Where a fluorescer is employed as the label, reading of the array may be accomplished by illuminating the array and reading the location and intensity of resulting fluorescence at each feature of the array. For example, a scanner may be used for this purpose where the scanner may be similar to, for example, the GENEARRAY scanner available from Agilent Technologies, Palo Alto, Calif. Other suitable apparatus and methods are described in U.S. patent applications: Ser. No. 09/846,125 “Reading Multi-Featured Arrays” by Dorsel, et al.; and Ser. No. 09/430,214 “Interrogating Multi-Featured Arrays” by Dorsel, et al. The relevant portions of these references are incorporated herein by reference. However, arrays may be read by methods or apparatus other than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere).

It is a significant feature of the present invention that the rate and extent of alteration of the first domain may be controlled by controlling the nature and concentration of the reactive agent in the fluid reagent and/or the nature of the first domain. For example, when the first domain comprises polyA, the number of subunits of A may be increased to increase the potential for reaction with the carboxylic acid and ultimately alteration of the first domain. On the other hand, the pKa of the acid may be lowered to increase the potential for the depurination reaction. For example, where the carboxylic acid is acetic acid, the number of halogen (chlorine, fluorine, bromine and iodine) substituents may be increased from 0 to 3 resulting in increased reactivity of the carboxylic acid in altering the polyA. Other types of systems may be controlled by controlling the respective first domains and/or reactive reagent that are employed. These will be suggested to one skilled in the art based on the disclosure herein and the knowledge of the skilled artisan.

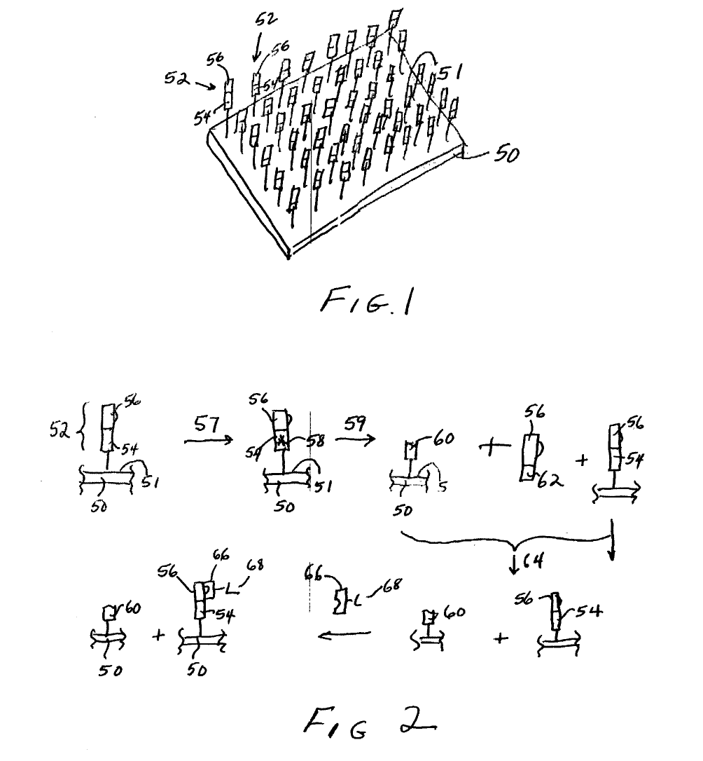

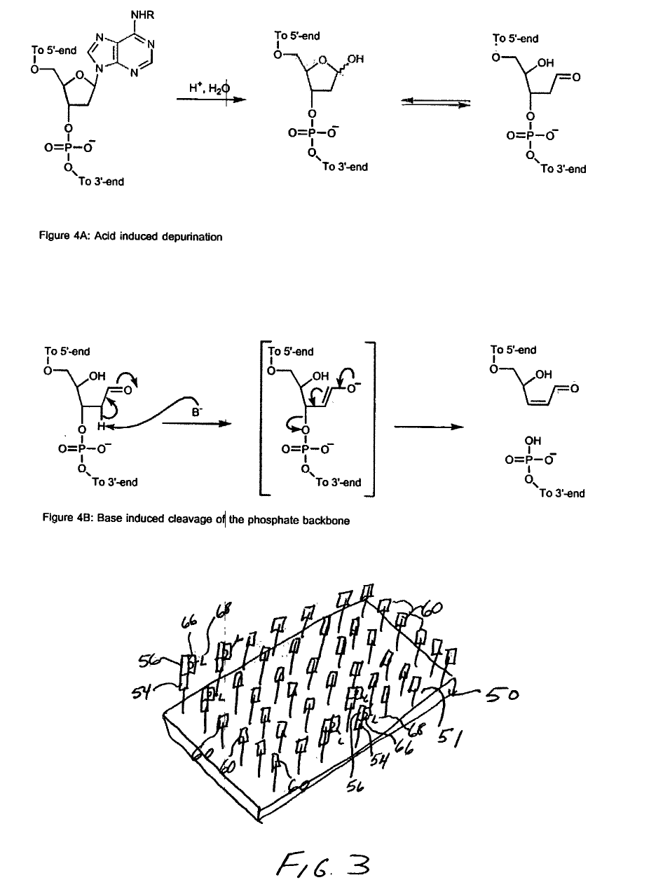

A particular example of the method as described above may be understood from the illustrations in FIGS. 1-3. It should be pointed out that the number of features shown on the surface of the support are only a small number of the actual number of such features that are normally present on the surface of the array. Support 50 has a plurality of test elements 52 bound to surface 51 of support 50. Each of test elements 52 comprises a first domain 54 and a second domain 56. Support 50 is placed into the chamber of a flow cell and a liquid reagent 57 is introduced therein by means of an inlet of the flow cell. The liquid reagent is reactive with first domain 54 to cause an alteration 58 in first domain 54. Subsequently, surface 51 is treated with a reagent 59 that promotes the cleavage of alterations 58 in first domains 54. As a result of the cleavage, different reaction products are formed, namely, a cleaved fragment 60 that remains bound to support 50 and a cleaved fragment 62 that comprises a portion of first domain 54 and second domain 56. Also included is intact first domain 54 and second domain 56 bound to surface 51 by means of first domain 54. As will be appreciated, the latter material results when no alteration of first domain 54 has taken place.

Subsequent to the treatment with cleaving reagent 59, the support is treated with wash reagent 64 to remove unbound materials such as the material comprising fragment 62 linked to first domain 56. Then, reagent 66, which comprises label 68 is added to surface 51. Reagent 66 specifically binds to second domain 56. As can be seen from FIG. 3, reagent 66 binds to only those sites at which second domain 56 is located, which are those sites where reagent 57 did not cause alteration of first domain 54. After removal of excess reagent 66, surface 51 is examined for the presence of signal from label 68. By examining the signal distribution on surface 51, the flow characteristics of, and reagent distribution in, reagent 57 may be determined.

Based on the determination described above, flow irregularities of a fluid in a chamber may be corrected. This may be accomplished by adjusting one or more fluid flow parameters of the chamber. The fluid flow parameter may be a flow characteristic of the fluid such as, for example, the overall fluid flow rate, peak centerline velocity, turbulence intensities, location of stagnant recirculation zones, and so forth. The fluid flow parameter may be an internal characteristic of the chamber such as, for example, gap thickness and location of the flow entrance and exit. Also, the overall flow cell geometry may be altered to increase or decrease its aspect ratio. Furthermore, flow conditioning may be required to homogenize the flow field over the span. In this case the flow would likely be introduced over a length of the flow cell edge to produce a uniform wall source to produce a unidirectional flow throughout the flow cell.

The present methods may be employed in the synthesis of a plurality of chemical compounds on supports with particular application to such synthesis on a commercial scale. Usually, the chemical compounds are those that are synthesized in a series of steps such as, for example, the addition of building blocks, which are chemical components of the chemical compound. Examples of such building blocks are those found in the synthesis of polymers. The apparatus and methods to which the present invention may have application are those that employ one or more flow cells, in which a different repetitive step in the synthesis of the chemical compounds is conducted.

As mentioned above, the chemical compounds are those that are synthesized in a series of steps, which usually involve linking together building blocks that form the chemical compound. The invention has particular application to the synthesis of oligomers or polymers. The oligomer or polymer is a chemical entity that contains a plurality of monomers. It is generally accepted that the term “oligomers” is used to refer to a species of polymers. The terms “oligomer” and “polymer” may be used interchangeably herein. Polymers usually comprise at least two monomers. Oligomers generally comprise about 5 to about 100 monomers, preferably, about 10 to about 50, more preferably about 15 to about 30 monomers. Examples of polymers include polydeoxyribonucleotides, polyribonucleotides, other polynucleotides that are Cglycosides of a purine or pyrimidine base, or other modified polynucleotides, polypeptides, polysaccharides, and other chemical entities that contain repeating units of like chemical structure. Exemplary of oligomers are oligonucleotides and peptides.

A monomer is a chemical entity that can be covalently linked to one or more other such entities to form an oligomer or polymer. Examples of monomers include nucleotides, amino acids, saccharides, peptoids, and the like and subunits comprising nucleotides, amino acids, saccharides, peptoids and the like. The subunits may comprise all of the same component such as, for example, all of the same nucleotide or amino acid, or the subunit may comprise different components such as, for example, different nucleotides or different amino acids. The subunits may comprise about 2 to about 2000, or about 5 to about 200, monomer units. In general, the monomers have first and second sites (e.g., C-termini and N-termini, or 5′ and 3′ sites) suitable for binding of other like monomers by means of standard chemical reactions (e.g., condensation, nucleophilic displacement of a leaving group, or the like), and a diverse element that distinguishes a particular monomer from a different monomer of the same type (e.g., an amino acid side chain, a nucleotide base, etc.). The initial substrate-bound, or support-bound, monomer is generally used as a building block in a multi-step synthesis procedure to form a complete ligand, such as in the synthesis of oligonucleotides, oligopeptides, oligosaccharides, etc. and the like.

A biomonomer references a single unit, which can be linked with the same or other biomonomers to form a biopolymer (for example, a single amino acid or nucleotide with two linking groups one or both of which may have removable protecting groups). A biomonomer fluid or biopolymer fluid reference a liquid containing either a biomonomer or biopolymer, respectively (typically in solution).

A biopolymer is a polymer of one or more types of repeating units. Biopolymers are typically found in biological systems and particularly include polysaccharides (such as carbohydrates), and peptides (which term is used to include polypeptides, and proteins whether or not attached to a polysaccharide) and polynucleotides as well as their analogs such as those compounds composed of or containing amino acid analogs or non-amino acid groups, or nucleotide analogs or non-nucleotide groups. This includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions.

Polynucleotides are compounds or compositions that are polymeric nucleotides or nucleic acid polymers. The polynucleotide may be a natural compound or a synthetic compound. Polynucleotides include oligonucleotides and are comprised of natural nucleotides such as ribonucleotides and deoxyribonucleotides and their derivatives although unnatural nucleotide mimetics such as 2′-modified nucleosides, peptide nucleic acids and oligomeric nucleoside phosphonates are also used. The polynucleotide can have from about 2 to 5,000,000 or more nucleotides. Usually, the oligonucleotides are at least about 2 nucleotides, usually, about 5 to about 100 nucleotides, more usually, about 10 to about 50 nucleotides, and may be about 15 to about 30 nucleotides, in length. Polynucleotides include single or multiple stranded configurations, where one or more of the strands may or may not be completely aligned with another.

A nucleotide refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. For example, a “biopolymer” includes DNA (including cDNA), RNA, oligonucleotides, and PNA and other polynucleotides as described in U.S. Pat. No. 5,948,902 and references cited therein (all of which are incorporated herein by reference), regardless of the source. An “oligonucleotide” generally refers to a nucleotide multimer of about 10 to 100 nucleotides in length, while a “polynucleotide” includes a nucleotide multimer having any number of nucleotides.

The support to which a plurality of chemical compounds is attached may be a support as described above. It is preferred in the present invention that the support used in the flow tests as discussed above be the same support that is employed in the synthesis of chemical compounds on the surface of the support.

The surface of a support is normally treated to create a primed or functionalized surface, that is, a surface that is able to support the synthetic steps involved in the production of the chemical compound. Functionalization relates to modification of the surface of a support to provide a plurality of functional groups on the support surface. The term “functionalized surface” is defined above. The manner of treatment is dependent on the nature of the chemical compound to be synthesized and on the nature of the support surface. In one approach a reactive hydrophilic site or reactive hydrophilic group is introduced onto the surface of the support. Such hydrophilic moieties can be used as the starting point in a synthetic organic process.

In one embodiment, the surface of the support, such as a glass support, is siliceous, i.e., comprises silicon oxide groups, either present in the natural state, e.g., glass, silica, silicon with an oxide layer, etc., or introduced by techniques well known in the art. The techniques for introducing siloxyl groups onto the surface of the support may be carried out in the same manner as discussed above. A procedure for the derivatization of a metal oxide surface is also described above.

The methods of the present invention are particularly useful in the synthesis of arrays of biopolymers. A biopolymer is a polymer of one or more types of repeating units relating to biology. Biopolymers are typically found in biological systems (although they may be made synthetically) and particularly include polysaccharides such as carbohydrates and the like, poly(amino acids) such as peptides including polypeptides and proteins, and polynucleotides, as well as such compounds composed of or containing amino acid analogs or non-amino acid groups, or nucleotide analogs or non-nucleotide groups. This includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions.

An array includes any one, two or three-dimensional arrangement of addressable regions bearing a particular chemical moiety or moieties such as, for example, biopolymers, e.g., one or more polynucleotides, associated with that region. An array is addressable in that it has multiple regions of different moieties, for example, different polynucleotide sequences, such that a region or feature or spot of the array at a particular predetermined location or address on the array can detect a particular target molecule or class of target molecules although a feature may incidentally detect non-target molecules of that feature.

The present methods and apparatus may be used in the synthesis of polypeptides. The synthesis of polypeptides involves the sequential addition of amino acids to a growing peptide chain. This approach comprises attaching an amino acid to the functionalized surface of the support. In one approach the synthesis involves sequential addition of carboxyl-protected amino acids to a growing peptide chain with each additional amino acid in the sequence similarly protected and coupled to the terminal amino acid of the oligopeptide under conditions suitable for forming an amide linkage. Such conditions are well known to the skilled artisan. See, for example, Merrifield, B. (1986), Solid Phase Synthesis, Sciences 232, 341-347. After polypeptide synthesis is complete, acid is used to remove the remaining terminal protecting groups.

The present invention has particular application to the synthesis of arrays of chemical compounds on a surface of a support. Typically, methods and apparatus of the present invention generate or use an array assembly that may include a support carrying one or more arrays disposed along a surface of the support and separated by inter-array areas. Normally, the surface of the support opposite the surface with the arrays does not carry any arrays. The arrays can be designed for testing against any type of sample, whether a trial sample, a reference sample, a combination of the foregoing, or a known mixture of components such as polynucleotides, proteins, polysaccharides and the like (in which case the arrays may be composed of features carrying unknown sequences to be evaluated). The surface of the support may carry at least one, two, four, or at least ten, arrays. Depending upon intended use, any or all of the arrays may be the same or different from one another and each may contain multiple spots or features of chemical compounds such as, e.g., biopolymers in the form of polynucleotides or other biopolymer. A typical array may contain more than ten, more than one hundred, more than one thousand or ten thousand features, or even more than one hundred thousand features, in an area of less than 20 cm² or even less than 10 cm². For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges.

Each feature, or element, within the molecular array is defined to be a small, regularly shaped region of the surface of the substrate. The features are arranged in a predetermined manner. Each feature of an array usually carries a predetermined chemical compound or mixtures thereof. Each feature within the molecular array may contain a different molecular species, and the molecular species within a given feature may differ from the molecular species within the remaining features of the molecular array. Some or all of the features may be of different compositions (for example, when any repeats of each feature composition are excluded the remaining features may account for at least 5%, 10%, or 20% of the total number of features). Each array may contain multiple spots or features and each array may be separated by spaces or areas. It will also be appreciated that there need not be any space separating arrays from one another. Interarray areas and interfeature areas are usually present but are not essential. These areas do not carry any chemical compound such as polynucleotide (or other biopolymer of a type of which the features are composed). Interarray areas and interfeature areas typically will be present where arrays are formed by the conventional in situ process or by deposition of previously obtained moieties, as described above, by depositing for each feature at least one droplet of reagent such as from a pulse jet (for example, an inkjet type head) but may not be present when, for example, photolithographic array fabrication processes are used. It will be appreciated though, that the interarray areas and interfeature areas, when present, could be of various sizes and configurations.

The devices and methods of the present invention are particularly useful in the synthesis of oligonucleotide arrays for determinations of polynucleotides. As explained briefly above, in the field of bioscience, arrays of oligonucleotide probes, fabricated or deposited on a surface of a support, are used to identify DNA sequences in cell matter. The arrays generally involve a surface containing a mosaic of different oligonucleotides or sample nucleic acid sequences or polynucleotides that are individually localized to discrete, known areas of the surface. In one approach, multiple identical arrays across a complete front surface of a single substrate or support are used.

Ordered arrays containing a large number of oligonucleotides have been developed as tools for high throughput analyses of genotype and gene expression. Oligonucleotides synthesized on a solid support recognize uniquely complementary nucleic acids by hybridization, and arrays can be designed to define specific target sequences, analyze gene expression patterns or identify specific allelic variations. The arrays may be used for conducting cell study, for diagnosing disease, identifying gene expression, monitoring drug response, determination of viral load, identifying genetic polymorphisms, analyze gene expression patterns or identify specific allelic variations, and the like.

The in situ synthesis of arrays of polynucleotides on the surface of a support usually involves attaching an initial nucleoside or nucleotide to a functionalized surface. The surface may be functionalized as discussed above. In one approach the surface is reacted with nucleosides or nucleotides that are also functionalized for reaction with the groups on the surface of the support. Methods for introducing appropriate amine specific or alcohol specific reactive functional groups into a nucleoside or nucleotide include, by way of example, addition of a spacer amine containing phosphoramidites, addition on the base of alkynes or alkenes using palladium mediated coupling, addition of spacer amine containing activated carbonyl esters, addition of boron conjugates, formation of Schiff bases.

After the introduction of the nucleoside or nucleotide onto the surface, the attached nucleotide may be used to construct the polynucleotide by means well known in the art. For example, in the synthesis of arrays of oligonucleotides, nucleoside monomers are generally employed. In this embodiment an array of the above compounds is attached to the surface and each compound is reacted to attach a nucleoside. Nucleoside monomers are used to form the polynucleotides usually by phosphate coupling, either direct phosphate coupling or coupling using a phosphate precursor such as a phosphite coupling. Such coupling thus includes the use of amidite (phosphoramidite), phosphodiester, phosphotriester, H-phosphonate, phosphite halide, and the like coupling.

One preferred coupling method is phosphoramidite coupling, which is a phosphite coupling. In using this coupling method, after the phosphite coupling is complete, the resulting phosphite is oxidized to a phosphate. Oxidation can be effected with iodine to give phosphates or with sulfur to give phosphorothioates. The phosphoramidites are dissolved in anhydrous acetonitrile to give a solution having a given ratio of amidite concentrations. The mixture of known chemically compatible monomers is reacted to a solid support, or further along, may be reacted to a growing chain of monomer units. In one particular example, the terminal 5′-hydroxyl group is caused to react with a deoxyribonucleoside-3′-O-(N,N-diisopropylamino)phosphoramidite protected at the 5′-position with dimethoxytrityl or the like. The 5′ protecting group is removed after the coupling reaction, and the procedure is repeated with additional protected nucleotides until synthesis of the desired polynucleotide is complete. For a more detailed discussion of the chemistry involved in the above synthetic approaches, see, for example, U.S. Pat. No. 5,436,327 at column 2, line 34, to column 4, line 36, which is incorporated herein by reference in its entirety.

Various ways may be employed to introduce the reagents for producing an array of polynucleotides on the surface of a support such as a glass support. Such methods are known in the art. One such method is discussed in U.S. Pat. No. 5,744,305 (Fodor, et al.) and involves solid phase chemistry, photolabile protecting groups and photolithography. Binary masking techniques are employed in one embodiment of the above. Arrays are fabricated in situ, adding one base pair at a time to a primer site. Photolithography is used to uncover sites, which are then exposed and reacted with one of the four base pair phosphoramidites. In photolithography the surface is first coated with a light-sensitive resist, exposed through a mask and the pattern is revealed by dissolving away the exposed or the unexposed resist and, subsequently, a surface layer. A separate mask is usually made for each pattern, which may involve four patterns for each base pair in the length of the probe.

Another in situ method employs inkjet printing technology to dispense the appropriate phosphoramidite reagents and other reagents onto individual sites on a surface of a support. Oligonucleotides are synthesized on a surface of a substrate in situ using phosphoramidite chemistry. Solutions containing nucleotide monomers and other reagents as necessary such as an activator, e.g., tetrazole, are applied to the surface of a support by means of thermal ink-jet technology. Individual droplets of reagents are applied to reactive areas on the surface using, for example, a thermal ink-jet type nozzle. The surface of the support may have an alkyl bromide trichlorosilane coating to which is attached polyethylene glycol to provide terminal hydroxyl groups. These hydroxyl groups provide for linking to a terminal primary amine group on a monomeric reagent. Excess of non-reacted chemical on the surface is washed away in a subsequent step. For example, see U.S. Pat. No. 5,700,637 and PCT WO 95/25116 and PCT application WO 89/10977.

Another approach for fabricating an array of biopolymers on a substrate using a biopolymer or biomonomer fluid and using a fluid dispensing head is described in U.S. Pat. No. 6,242,266 (Schleifer, et al.). The head has at least one jet that can dispense droplets onto a surface of a support. The jet includes a chamber with an orifice and an ejector, which, when activated, causes a droplet to be ejected from the orifice. Multiple droplets of the biopolymer or biomonomer fluid are dispensed from the head orifice so as to form an array of droplets on the surface of the substrate.

In another embodiment (U.S. Pat. No. 6,232,072) (Fisher) a method of, and apparatus for, fabricating a biopolymer array is disclosed. Droplets of fluid carrying the biopolymer or biomonomer are deposited onto a front side of a transparent substrate. Light is directed through the substrate from the front side, back through a substrate back side and a first set of deposited droplets on the first side to an image sensor.

An example of another method for chemical array fabrication is described in U.S. Pat. No. 6,180,351 (Cattell). The method includes receiving from a remote station information on a layout of the array and an associated first identifier. A local identifier is generated corresponding to the first identifier and associated array. The local identifier is shorter in length than the corresponding first identifier. The addressable array is fabricated on the substrate in accordance with the received layout information.

Other methods for synthesizing arrays of oligonucleotide on a surface include those disclosed by Gamble, et al., WO97/44134; Gamble, et al., WO98/10858; Baldeschwieler, et al., WO95/25116; Brown, et al., U.S. Pat. No. 5,807,522; and the like.

In general, in the above synthetic steps involving monomer addition such as, for example, the phosphoramidite method, there are certain repetitive steps that are carried out in one or more flow cells. Such steps include, e.g., washing the surface of the support prior to or after a reaction, oxidation of substances such as oxidation of a phosphite group to a phosphate group, removal of protecting groups, blocking of sites to prevent reaction at such site, capping of sites that did not react with a phosphoramidite reagent, deblocking, and so forth. In addition, under certain circumstances other reactions may be carried out in a flow cell such as, for example, phosphoramidite monomer addition, modified phosphoramidite addition, other monomer additions, addition of a polymer chain to a surface for linking to monomers, and so forth.

For in situ fabrication methods, multiple different reagent droplets are deposited by pulse jet or other means at a given target location in order to form the final feature (hence a probe of the feature is synthesized on the array substrate). The in situ fabrication methods include those described in U.S. Pat. No. 5,449,754 for synthesizing peptide arrays, and in U.S. Pat. No. 6,180,351 and WO 98/41531 and the references cited therein for polynucleotides, and may also use pulse jets for depositing reagents. The in situ method for fabricating a polynucleotide array typically follows, at each of the multiple different addresses at which features are to be formed, the same conventional iterative sequence used in forming polynucleotides from nucleoside reagents on a support by means of known chemistry. This iterative sequence can be considered as multiple ones of the following attachment cycle at each feature to be formed: (a) coupling an activated selected nucleoside (a monomeric unit) through a phosphite linkage to a functionalized support in the first iteration, or a nucleoside bound to the substrate (i.e. the nucleoside-modified substrate) in subsequent iterations; (b) optionally, blocking unreacted hydroxyl groups on the substrate bound nucleoside (sometimes referenced as “capping”); (c) oxidizing the phosphite linkage of step (a) to form a phosphate linkage; and (d) removing the protecting group (“deprotection”) from the now substrate bound nucleoside coupled in step (a), to generate a reactive site for the next cycle of these steps. The coupling can be performed by depositing drops of an activator and phosphoramidite at the specific desired feature locations for the array. Capping, oxidation and deprotection can be accomplished by treating the entire substrate (“flooding”) with a layer of the appropriate reagent. The functionalized support (in the first cycle) or deprotected coupled nucleoside (in subsequent cycles) provides a substrate bound moiety with a linking group for forming the phosphite linkage with a next nucleoside to be coupled in step (a). Final deprotection of nucleoside bases can be accomplished using alkaline conditions such as ammonium hydroxide, in another flooding procedure in a known manner. Conventionally, a single pulse jet or other dispenser is assigned to deposit a single monomeric unit.

The foregoing chemistry of the synthesis of polynucleotides is described in detail, for example, in Caruthers, Science 230: 281-285, 1985; Itakura, et al., Ann. Rev. Biochem. 53: 323-356; Hunkapillar, et al., Nature 310: 105-110, 1984; and in “Synthesis of Oligonucleotide Derivatives in Design and Targeted Reaction of Oligonucleotide Derivatives”, CRC Press, Boca Raton, Fla., pages 100 et seq., U.S. Pat. Nos. 4,458,066, 4,500,707, 5,153,319, 5,869,643 and European patent application, EP 0294196, and elsewhere. The phosphoramidite and phosphite triester approaches are most broadly used, but other approaches include the phosphodiester approach, the phosphotriester approach and the H-phosphonate approach. The substrates are typically functionalized to bond to the first deposited monomer. Suitable techniques for functionalizing substrates with such linking moieties are described, for example, in Southern, E. M., Maskos, U. and Elder, J. K., Genomics, 13, 1007-1017, 1992.

In the case of array fabrication, different monomers and activator may be deposited at different addresses on the substrate during any one cycle so that the different features of the completed array will have different desired biopolymer sequences. One or more intermediate further steps may be required in each cycle, such as the conventional oxidation, capping and washing steps in the case of in situ fabrication of polynucleotide arrays (again, these steps may be performed in flooding procedure).

Some or all of the above steps may be performed using flow cells that have been tested in accordance with the present invention. Accordingly, for example, after addition of a nucleoside monomer, whether using an ink jet method, a photolithography method or the like, the support is placed into a chamber of a flow cell. The flow cell allows fluids to be passed into the chamber where the support is disposed. The flow parameters for the liquid reagents introduced into the flow cell are adjusted based on the information obtained from a method in accordance with the present invention. The nature of the chamber and mounting of the support inside the chamber are as discussed above.

The inlet of the flow cell is usually in fluid communication with an element that controls the flow of fluid into the flow cell such as, for example, a manifold, a valve, and the like or combinations thereof. A controller communicating with a computer may be used to adjust the flow of reagent into the chamber by controlling the valves employed to introduce the reagent. This element in turn is in fluid communication with one or more fluid reagent dispensing stations. In this way different fluid reagents for one step in the synthesis of the chemical compound may be introduced sequentially into the flow cell. These reagents may be, for example, a chemical reagent that forms part of the chemical compound by addition thereto, wash fluids, oxidizing agents, reducing agents, blocking or protecting agents, unblocking (deblocking) or deprotecting agents, and so forth. Any reagent that is normally a solid reagent may be converted to a fluid reagent by dissolution in a suitable solvent, which may be a protic solvent or an aprotic solvent as discussed above. In one embodiment the fluid dispensing stations are affixed to a base plate or main platform to which the flow cells are mounted. Any fluid dispensing station may be employed that dispenses fluids such as discussed above.

Upon completion of the first step in the synthesis of the chemical compound, the support may be treated with another reagent after suitable washing of the surface. Alternatively, the support may be removed from the flow cell and transferred to a second flow cell, which generally has the same or similar configuration as the first flow cell but need not. The support is transported by a transfer element such as a robotic arm, and so forth. In one embodiment a transfer robot is mounted on the main platform of the present apparatus. The transfer robot may comprise a base, an arm that is movably mounted on the base, and an element for grasping the support during transport that is attached to the arm. The element for grasping the support may be, for example, movable finger-like projections, and the like. In use, the robotic arm is activated so that the support is grasped by the above-mentioned element. The arm of the robot is moved so that the support is delivered to the second flow cell, which is in the open position so that the support is delivered into the chamber thereof. The second flow cell is operated in substantially the same manner as described above for the first flow cell to carry out a second step in the synthesis of the chemical compound.

The support may be transferred to additional flow cells to complete the synthesis of the chemical compound. It is within the purview of the present invention that one or more steps in the synthesis process is a repeat of an earlier step because the chemical component that is to be added to the growing molecule is the same as that in a previous step. In this instance the transfer element delivers the support to a flow cell in which the earlier repetitive step was carried out and at which the dispensing stations have the necessary reagents for conducting this step.

The amount of the reagents employed in each synthetic step in the method of the present invention is dependent on the nature of the reagents, solubility of the reagents, reactivity of the reagents, availability of the reagents, purity of the reagents, and so forth. Such amounts should be readily apparent to those skilled in the art in view of the disclosure herein. Usually, stoichiometric amounts are employed, but excess of one reagent over the other may be used where circumstances dictate. Typically, the amounts of the reagents are those necessary to achieve the overall synthesis of the chemical compound in accordance with the present invention. The time period for conducting the present method is dependent upon the specific reaction and reagents being utilized and the chemical compound being synthesized.

As mentioned above, a different flow cell may be employed for each distinct repetitive step. Using as an example the synthesis of polynucleotides on a surface by the phosphoramidite method, the step of oxidation of phosphite to phosphate is carried out in a dedicated flow cell. Accordingly, following addition of a monomer, the support is placed in the flow cell, which is then closed to form a liquid tight seal. Various fluid dispensing stations are connected by means of a manifold and suitable valves to the inlet of the flow cell. Each of the fluid dispensing stations contains a different fluid reagent involved in performing the particular synthetic addition of monomer. Thus, in this example, one station may contain an oxidizing agent for oxidizing the phosphite to the phosphate and another station may contain a wash reagent such as acetonitrile.

In all of the steps mentioned below by way of example, the flow parameters of the liquid reagents utilized are adjusted based on the results obtained by carrying out the method of the invention on each flow cell. The present invention may be applied to each flow cell employed in the synthesis of the oligonucleotides and the flow parameters and/or reagent concentration for each step may be adjusted accordingly. The wash reagent is first allowed to pass into and out of the flow cell. Next, the oxidizing agent is allowed to pass into and out of the flow cell and the surface is again washed with the wash reagent as described above. The support is then transported from this first flow cell to a second flow cell. At this point, a deblocking reagent for removing a protecting group is allowed to pass into and out of the second flow cell. The deblocking reagent is contained in a fluid dispensing station that is in fluid communication with the second flow cell. Next, wash reagent contained in a fluid dispensing station that is in fluid communication with the second flow cell is passed into and out of the second flow cell. Following the above synthetic steps, the support is transported from the second flow cell to a station where the next monomer addition is carried out and the above repetitive synthetic steps are conducted in the first and second flow cells as discussed above.

An apparatus for synthesizing an array of biopolymers on the surface of a support may comprise a platform and a plurality of flow cells mounted on the platform. The flow cells comprise a chamber, a holder for the support, at least one inlet and an outlet, wherein each of the inlets is in fluid communication with a manifold. One or more fluid dispensing stations are mounted on the platform and are in fluid communication with one or more of the plurality of flow cells by means of the manifolds. A station for monomer addition to the surface of the support is mounted on the platform. The apparatus also comprises a mechanism for moving a support to and from the station for monomer addition and a flow cell and from one flow cell to another flow cell. The mechanism may be, for example, a robotic arm, and so forth.

In one embodiment of a mechanism for moving a support from one flow cell to another flow cell, the support is delivered into the opening in the wall of the flow cell housing by engagement with a holding element, which usually comprises a main arm and an end portion that contacts and engages a surface of the support. In one embodiment the holding element is in the form of a fork that is vacuum activated. Other embodiments of the holding element include, for example, grasping elements such as movable finger-like projections, and the like. The holding element is usually part of a transfer robot that comprises a robotic arm that is capable or transferring the support from various positions where steps in the synthesis of the chemical compound are performed such as between several flow devices in accordance with the present invention. In one embodiment a transfer robot is mounted on the main platform. The transfer robot may comprise a base, an arm that is movably mounted on the base, and an element for holding the support during transport that is attached to the arm. Also included is a controller for controlling the movement of the mechanism. The apparatus may further comprise a sensor in fluid communication with holding chamber.

The apparatus further comprise appropriate electrical and mechanical architecture and electrical connections, wiring and devices such as timers, clocks, and so forth for operating the various elements of the apparatus. Such architecture is familiar to those skilled in the art and will not be discussed in more detail herein.

The methods in accordance with the present invention may be carried out under computer control, that is, with the aid of a computer. For example, an IBM® compatible personal computer (PC) may be utilized. The computer is driven by software specific to the methods described herein. A preferred computer hardware capable of assisting in the operation of the methods in accordance with the present invention involves a system with at least the following specifications: Pentium® processor or better with a clock speed of at least 100 MHz, at least 32 megabytes of random access memory (RAM) and at least 80 megabytes of virtual memory, running under either the Windows 95 or Windows NT 4.0 operating system (or successor thereof).

Software that may be used to carry out the methods may be, for example, Microsoft Excel or Microsoft Access, suitably extended via user-written functions and templates, and linked when necessary to stand-alone programs. Examples of software or computer programs used in assisting in conducting the present methods may be written, preferably, in Visual BASIC, FORTRAN and C⁺⁺. It should be understood that the above computer information and the software used herein are by way of example and not limitation. The present methods may be adapted to other computers and software. Other languages that may be used include, for example, PASCAL, PERL or assembly language.

A computer program may be utilized to carry out the above method steps. The computer program provides for (i) placing a support into a chamber of a first flow device, (ii) utilizing the flow parameters ascertained by applying the method of the invention to the flow cell, introducing a fluid reagent for conducting a reaction step into the reagent chamber, (iii) removing the fluid reagent from the reagent chamber, (iv) removing the support from the housing chamber, (v) placing the support into a chamber of a flow device, (vi) utilizing the flow parameters ascertained by applying the method of the invention to the flow cell, introducing a fluid reagent for conducting a reaction step into the reagent chamber, (vii) removing the fluid reagent from the reagent chamber, (viii) removing the support from the housing chamber. The computer program may provide for moving the support to and from a station for monomer addition at a predetermined point in the aforementioned method.

Another aspect of the present invention is a computer program product comprising a computer readable storage medium having a computer program stored thereon which, when loaded into a computer, performs the aforementioned method.

Following receipt by a user of an array made utilizing the principles of the present invention, it will typically be exposed to a sample (for example, a fluorescent-labeled polynucleotide or protein containing sample) and the array is then read. Reading of the array may be accomplished by illuminating the array and reading the location and intensity of resulting fluorescence at each feature of the array. For example, a scanner may be used for this purpose where the scanner may be similar to, for example, the AGILENT MICROARRAY SCANNER available from Agilent Technologies Inc, Palo Alto, Calif. Other suitable apparatus and methods are described in U.S. patent application Ser. No. 09/846,125 “Reading Multi-Featured Arrays” by Dorsel, et al.; and Ser. No. 09/430,214 “Interrogating Multi-Featured Arrays” by Dorsel, et al. The relevant portions of these references are incorporated herein by reference. However, arrays may be read by methods or apparatus other than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). Results from the reading may be raw results (such as fluorescence intensity readings for each feature in one or more color channels) or may be processed results such as obtained by rejecting a reading for a feature that is below a predetermined threshold and/or forming conclusions based on the pattern read from the array (such as whether or not a particular target sequence may have been present in the sample). The results of the reading (processed or not) may be forwarded (such as by communication) to a remote location if desired, and received there for further use (such as further processing).

When one item is indicated as being “remote” from another, this is referenced that the two items are at least in different buildings, and may be at least one mile, ten miles, or at least one hundred miles apart. “Communicating” information references transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data.

As mentioned above, another embodiment of the invention is a kit comprising in packaged combination (a) a support to which is bound a plurality of test elements, each of the test elements comprising a reaction domain and a detection domain, and (b) one or more reagents reactive with the reaction domain such as, for example, a cleavage reagent. Optionally, the kit may comprise reagents for binding with the detection domain. The reagent(s) reactive with the reaction domain such as, e.g., a cleavage reagent, may be in solution. The kit may further include a hybridization kit for conducting hybridization reactions. The kit may further include a dye for the detection step. The kit may further include a housing for holding the support in a flow chamber. The kit may also include a written description of a method in accordance with the present invention and instructions for carrying out such method.

One specific embodiment of the invention is a kit comprising in packaged combination:

(a) a support to which is bound a plurality of test elements, each of said test elements comprising a reaction domain and a detection domain, and

(b) a reagent reactive with a reaction domain. In a kit according to the above, the reaction domains may comprise nucleotides such as, for example, (N)ₙ wherein N is a nucleotide and n is about 5 to about 50. In a specific embodiment N is A. In a kit according to the above, the detection domains may comprise a member of a specific binding pair such as, for example, polypeptides and polynucleotides. In a kit according to the above the reagent reactive with a reaction domain may be a cleavage agent.

Other Specific Embodiments Include:

A method for determining a functional property of a fluid in a chamber, said method comprising:

(a) introducing into said chamber a support to which is bound a plurality of test elements, each of said test elements comprising a reaction domain and a detection domain,

(b) introducing into said chamber a fluid that is interactive with said reaction domains,

(c) removing said fluid from said chamber, and

(d) determining by means of said detection domains the locations at which said fluid has not interacted with said reaction domains and relating said locations to the functional property of said fluid.

A method according to the above wherein said fluid is a gas.

A method according to the above wherein said fluid is a liquid.

A method according to the above wherein said reaction domains comprise nucleotides.

A method according to the above wherein said reaction domains comprise (N)ₙ wherein N is a nucleotide and n is about 5 to about 50.

A method according to the above wherein N is A.

A method according to the above wherein said detection domains comprise a member of a specific binding pair.

A method according to the above wherein said member is selected from the group consisting of polypeptides and polynucleotides.

A method according to the above wherein said determining of step (d) comprises treating said test elements to modify only those reaction domains that have interacted with said fluid.

A method according to the above wherein said treating comprises exposing said test elements to a cleavage reagent that cleaves only those reaction domains that have interacted with said fluid.

A method according to the above wherein said method further comprises exposing said support to a complementary member of said specific binding pair wherein said complementary member comprises a detectable label.

A method according to the above wherein said member is a polynucleotide and said method further comprises adding to said support a complementary polynucleotide comprising a detectable label.

A method according to the above wherein said member of a specific binding pair comprises a detectable label and said method comprises examining said support for the location of said detectable labels subsequent to said cleaving.

A method according to the above wherein said detectable label is selected from the group consisting of fluorescent, phosphorescent, and chemiluminescent compounds, radioisotopes, enzymes.

A method according to the above wherein said detectable label is selected from the group consisting of fluorescent, phosphorescent, and chemiluminescent compounds, radioisotopes, enzymes.

A method according to the above wherein said functional property is selected from the group consisting of the flow pattern of said fluid, reagent distribution within said fluid, and time dependent reactivity of said fluid.

A method according to the above, said method further comprising the step of using results of said determining to adjust the flow parameters for introducing fluid into said chamber.

### EXAMPLES

The invention is demonstrated further by the following illustrative examples. Parts and percentages are by weight unless otherwise indicated. Temperatures are in degrees Centigrade (° C.) unless otherwise specified. The following preparations and examples illustrate the invention but are not intended to limit its scope.

### Example 1

An experimental array containing 33,820 features on a 3×3 inch glass wafer was prepared by in situ coupling of phosphoramidite reagents deposited by an inkjet-based apparatus using standard DNA synthesis and standard DNA synthesis reagents. The DNA synthesis cycle was repeated appropriately to obtain 25-mer oligonucleotides within each feature. 3,020 of the features contained the same test element of sequence 3′-AAAAAAAAAAAAAAAAAATCTCCCA-5′ (SEQ ID NO:1) (reaction domain is in bold, detection domain is in italic) while the remaining oligonucleotides were internal, positive and negative controls. The standard synthesis of the array was altered by stopping the fabrication prior to the ultimate deprotection of the base protection groups.

### Example 2

An experimental array can also be prepared with the following modifications the preparation described in Example 1. Pre-synthesized 5′-end labeled and 3′-end modified oligonucleotides were deposited by inkjet printing and not synthesized in situ on the glass surfaces. DNA attachment to the derivatized surface was performed by a coupling reaction such as by reaction between an amino group terminated oligonucleotide and an aldehyde functionalized substrate to form an imine. The sequence of the oligonucleotide was 3′-NH₂-AAAAAAAAAAAAAAAAAA-Cy3/Cy5-5′ (SEQ ID NO:2).

### Example 3

To perform the experimental flow visualization of a liquid in an uncharacterized flow cell containing an inlet and an outlet, an experimental array, such as from Examples 1 or 2, was placed against the flow cell and its position registered for subsequent data analysis. By the term “flow visualization” is meant the flow patterns inferred from the amount of chemical reaction having occurred on the surface. It was assumed that there was sufficient temporal resolution to provide a measurable gradient in the final reaction quality over the test surface. This being the case, the gradients across the cell recorded the time-averaged history of the diffusion of reactant to the surface. The spatial gradient depended on the reaction rate and diffusion coefficient for the system through the Biot number, Bi=

$$Bi = \frac{kl}{D} .$$

kl/D. Here k is the pseudo first order reaction coefficient for a heterogeneous reaction, l is a characteristic length scale and D is the diffusion coefficient for the reactant in the fluid within the flow cell. This form of the Biot number is also sometimes referred to as the second Damkohler number.

This method is superior to introducing a passive scalar such as a dye into the flow since a dye introduced into the bulk will not likely allow one to infer the efficacy of mixing or diffusion of reactant through the thin, slow-moving, viscous sub-layer near the surface. In the present invention, the resulting signal measurement gives a true indication of the amount of reactant diffusing to and reacting with the surface.

A solution containing 2% dichloroacetic acid in toluene was introduced through the inlet and was then removed through the outlet after a reaction time (waiting time). In some cases, the reaction was repeated by cycling through the injection, waiting and removal cycles as many times as necessary. After reaction in the flow cell, the array was removed, washed once with acetonitrile, then once with water, and finally twice with acetonitrile. Subsequently, the array was dried and reacted with ethanolamine for 30 min at room temperature to achieve both cleavage of the depurinated nucleosides and removal of the base protecting groups. After rinsing in deionized water and drying, the array was diced in two 1×3 inches slides.

### Example 4

The imaging of a flow visualization experiment described in example 3, using and array prepared in example 1 was performed by the hybridization of the experimental array with an excess of a mixture of Cy3/Cy5 labeled oligonucleotides complementary to the detection domain (5′-Cy3/Cy5-TAGAGGGT-3′) and, if necessary, to other sequences present on the array. The hybridization buffer, temperature, duration and washing conditions were those recommended in the Agilent hybridization kit (Agilent Technologies Inc., Palo Alto, Calif.). Fluorescent detection of the hybridized, labeled target was performed on a G2565AA Agilent DNA microarray scanner (Agilent Technologies Inc., Palo Alto, Calif.) and data analysis was performed using Access, Excel, Spotfire and standard user created macros to average and display the data.

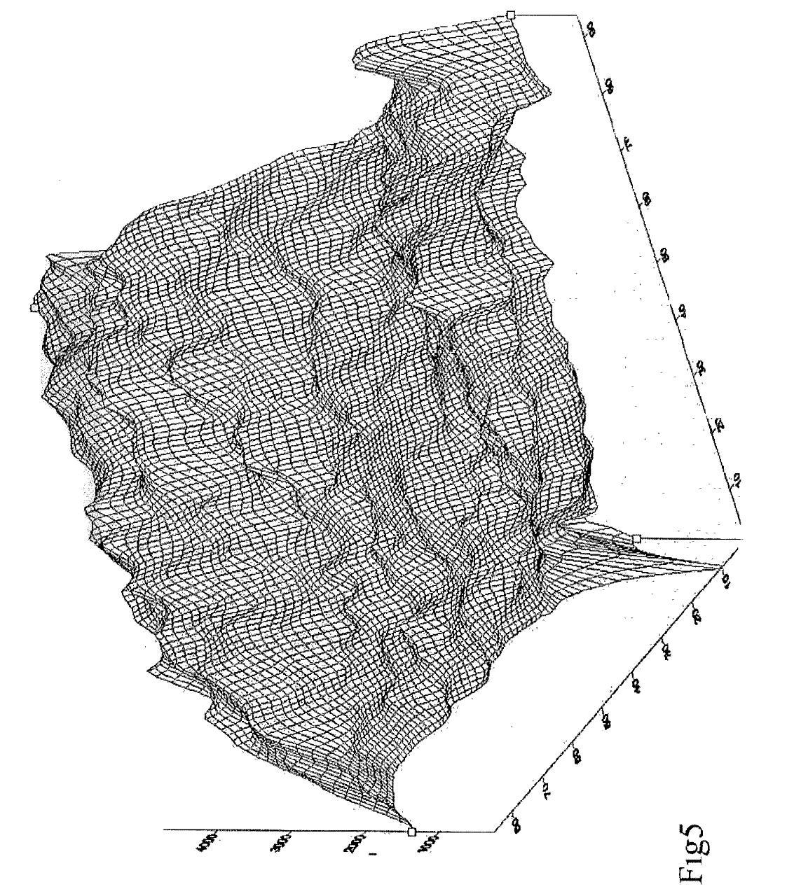

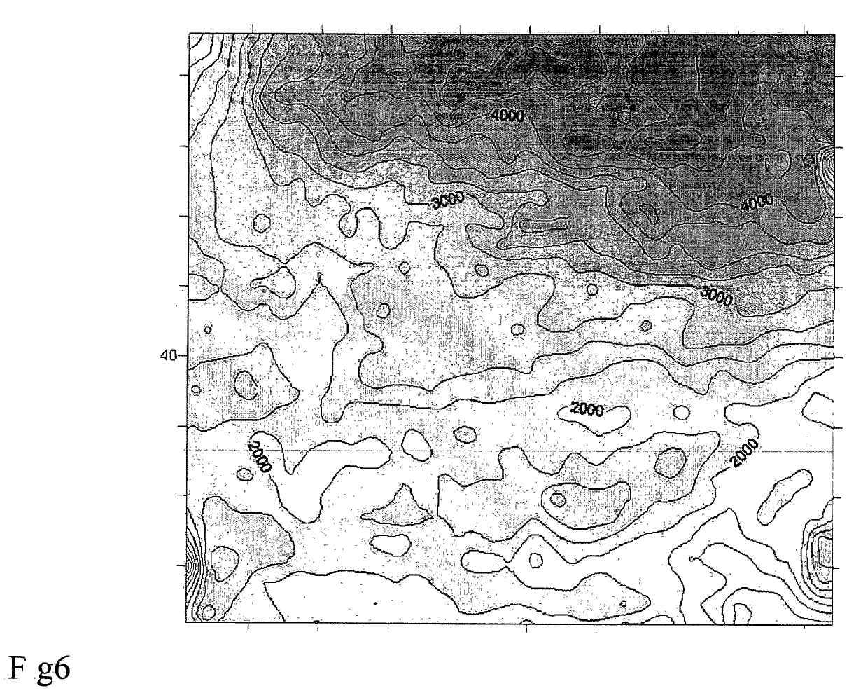

The average signal intensity of each feature containing the detection domain was placed on a grid and plotted in 3 dimensions as a function of its position on the arrays (FIG. 5). The data points of highest signal (z axis) correspond to the location of the lowest extent of reaction with the reaction domain inside the flow cell. FIG. 6 represents the two dimensional, spatial distribution of reagents within the flow cell (the hybridization signal is shown by a gray scale). As seen in FIGS. 5 and 6, large, spatial, signal variations are detected in this flow cell indicating a non-uniform flow distribution. The exact location affected in the flow cell can be inferred from the registration marks used during the experiment. From the information obtained in this experiment as depicted in FIGS. 5 and 6, appropriate action can be taken to modify the flow cell and/or flow pattern and obtain uniform reaction distribution.

### Example 5

The imaging of a flow visualization experiment described in Example 3, using and array prepared in Example 2 was performed as detailed in Example 4, with the notable exception that no hybridization was required since the attached oligonucleotides were already fluorescent labeled.

### Example 6

The experimental flow visualization of a gas in an uncharacterized flow cell containing an inlet and an outlet was performed as in Example 3, with the following modifications. Instead of 2% dichloroacetic acid in toluene, trifluoroacetic acid in nitrogen was introduced through the inlet and was then removed through the outlet after a reaction time (residence time). The trifluoroacetic acid was dispersed in nitrogen by bubbling nitrogen in a solution of trifluoroacetic acid in toluene. The acid concentration in nitrogen may be controlled by variation of the nitrogen flow rate, temperature, bubble size and/or trifluoroacetic acid concentration in toluene.

### Example 7

The methods described in Examples 1 through 6 to characterize the flow distribution within a flow cell may be performed for any flow cell. Accordingly, the methods may be applied in situations other than the synthesis of DNA microarrays, such as, for example, the synthesis of peptide microarrays. Theflow cell should be geometrically similar and the relevant non-dimensional groups such as the Reynolds number Re=

$$Re = \frac{ρ ul}{μ}$$

Pul (liquid or gas) employed in the normal utilization should match those of the fluid used during characterization. Here, ρ is the fluid density, u is the characteristic fluid speed, μ is the dynamic viscosity and l is a length scale that characterizes the flow. For flow cells, l is likely the gap thickness. For a flow involving a free surface, the Weber number We=

$$We = \frac{ρ {u}^{2} l}{γ}$$

and perhaps the Froude Fr=

$$Fr = \frac{{u}^{2}}{gl}$$

numbers should be matched where, ≢ is the surface tension and g is gl acceleration due to gravity. Examples include systems where bubble mixing is utilized or a liquid-filled chamber is purged and refilled. The Reynolds and Weber numbers of the flow in Example 3 and 7 were, therefore, matched as necessary by varying the viscosity and surface tension of the solvent and/or by the addition of material properties modifiers such as, for example, thickeners such as, e.g., glycerin, PEG, etc., and surfactants such as, e.g., sodium laurel sulfate, a TRITON X® surfactant, TWEEN 20®, a tergitol surfactant, and the like. Also, the Reynolds Froude and Weber numbers may be altered by altering physical characteristics such as changing the velocity and characteristic length scale.

In view of the above, it should be apparent that the present invention provides for the characterization of a new fluid in a known flow cell, a known fluid in a new flow cell, or a new fluid/flowcell combination.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims. Furthermore, the foregoing description, for purposes of explanation, used specific nomenclature to provide a thorough understanding of the invention. However, it will be apparent to one skilled in the art that the specific details are not required in order to practice the invention. Thus, the foregoing descriptions of specific embodiments of the present invention are presented for purposes of illustration and description; they are not intended to be exhaustive or to limit the invention to the precise forms disclosed. Many modifications and variations are possible in view of the above teachings. The embodiments were chosen and described in order to explain the principles of the invention and its practical applications and to thereby enable others skilled in the art to utilize the invention.

What is claimed is:

1. A method for determining a functional property of a fluid in a chamber, said method comprising:
(a) introducing into said chamber a support to which is bound a plurality of test elements, each of said test elements comprising a reaction domain and a detection domain,
(b) introducing into said chamber a fluid that is interactive with said reaction domains,
(c) removing said fluid from said chamber, and
(d) determining by means of said detection domains the locations at which said fluid has not interacted with said reaction domains and relating said locations to the functional property of said fluid.

2. A method according to claim 1 wherein said reaction domains comprise nucleotides.

3. A method according to claim 1 wherein said detection domains comprise a member of a specific binding pair.

4. A method according to claim 1 wherein said determining of step (d) comprises treating said test elements to modify only those reaction domains that have interacted with said fluid.

5. A method according to claim 1 wherein said functional property is selected from the group consisting of the flow pattern of said fluid, reagent distribution within said fluid, and time dependent reactivity of said fluid.

6. A method according to claim 1, said method further comprising the step of using results of said determining to adjust the flow parameters for introducing fluid into said chamber.

7. A method for determining the flow pattern and/or reagent distribution of a liquid reagent in a chamber, said method comprising:
(a) introducing into said chamber a support to which is bound a plurality of test elements, each of said test elements comprising (i) a first portion proximal said support, said first portion comprising (N)n wherein N is a nucleotide and n is about 5 to about 50, and (ii) a second portion distal said support, said second portion comprising a polynucleotide,
(b) introducing into said chamber said liquid reagent that is reactive with said reaction domains,
(c) removing said liquid reagent from said chamber,
(d) exposing said support to a cleavage reagent that cleaves only those first portions that have reacted with said liquid reagent,
(e) exposing said support a complementary polynucleotide comprising a detectable label,
(f) examining said support for the locations at which said detectable label is present and relating said locations to the flow pattern and/or the reagent distribution of said liquid reagent in said chamber.

8. A method according to claim 7 wherein N is A.

9. A method according to claim 7 wherein said liquid reagent is a reagent that causes the depurination of said first portion.

10. A method according to claim 7 wherein said liquid reagent is an acid.

11. A method according to claim 7 wherein said cleavage reagent is a base.

12. A method according to claim 7 wherein the reactivity of said first portion is adjusted by adjusting the length n of (N)ₙ.

13. A method according to claim 7 wherein said detectable label is selected from the group consisting of fluorescent, phosphorescent, and chemiluminescent compounds, radioisotopes, enzymes.

14. A method according to claim 7 wherein said chamber comprises at least one inlet and an outlet and a holder for said support.

15. A method according to claim 7, said method further comprising the step of using results of said determining to adjust the flow parameters for introducing fluid into said chamber.

16. A method for synthesizing an array of biopolymers on the surface of a support wherein said synthesis comprises a plurality of monomer additions, said method comprising after each of said monomer additions:
(a) placing said support into a chamber subjected to the method according to claim 7 and subsequently subjected to at least one wash step, and
(b) subjecting said surface of said support in said chamber to a step of said synthesis that is subsequent to a monomer addition.

17. A method according to claim 16 wherein said biopolymers are polynucleotides.

18. A method according to claim 16 wherein said step of said synthesis is selected from the group consisting of (i) subjecting said surface to an oxidizing agent, (ii) subjecting said surface to an agent for removing a protecting group and (iii) flowing a liquid reagent comprising an organic solvent into said chamber.

19. A method according to claim 16 wherein said biopolymers are synthesized on said surface in multiple arrays and said support is subsequently diced into individual arrays of biopolymers on a support.

20. A method according to claim 19 further comprising exposing the array to a sample and reading the array.

21. A method according to claim 20 comprising forwarding data representing a result obtained from a reading of the array.

22. A method according to claim 21 wherein the data is transmitted to a remote location.

23. A method according to claim 21 comprising receiving data representing a result of an interrogation obtained by the reading of the array.

24. A method for synthesizing an array of biopolymers on the surface of a support, said method comprising:
(a) placing said support into a reaction chamber and applying to said surface said biopolymers or precursors of said biopolymers,
(b) removing said support from said reaction chamber and placing said support into a flow chamber wherein said flow chamber has previously been subjected to a method according to claim 1 and to at least one wash step,
(c) flowing into said flow chamber a liquid reagent for carrying out the synthesis of said biopolymers wherein the flow parameters of said flowing are adjusted based on the results of the determination of claim 1,
(d) removing said support from said flow chamber and
(e) repeating steps (a)-(c) to form said array of biopolymers.

25. A method according to claim 24 wherein said biopolymers are polynucleotides.

26. A method according to claim 24 wherein said step of said synthesis is selected from the group consisting of (i) subjecting said surface to an oxidizing agent and (ii) subjecting said surface to an agent for removing a protecting group.

27. A method for correcting for flow irregularities of a fluid in a chamber, said method comprising:
(a) introducing into said chamber a support to which is bound a plurality of test elements, each of said test elements comprising a reaction domain and a detection domain,
(b) introducing into said chamber a fluid that is interactive with said reaction domains,
(c) removing said fluid from said chamber,
(d) determining by means of said detection domains the locations at which said fluid has not interacted with said reaction domains and relating said locations to the functional property of said fluid, and
(e) adjusting one or more fluid flow parameters of said chamber based on the determination of step (d).

28. A method according to claim 27 wherein said fluid flow parameter is a flow characteristic of the fluid or an internal characteristic of said chamber.

29. A method according to claim 27 wherein said fluid is a liquid.

30. A method according to claim 27 wherein said reaction domains comprise nucleotides.

31. A method according to claim 27 wherein said reaction domains comprise (N)ₙ wherein N is a nucleotide and n is about 5 to about 50.

32. A method according to claim 31 wherein N is A.

33. A method according to claim 27 wherein said detection domains comprise a member of a specific binding pair.

34. A method according to claim 33 wherein said member is selected from the group consisting of polypeptides and polynucleotides.

35. A kit comprising in packaged combination:
(a) a support comprising a plurality of features on a surface of said support, said features comprising (i) a first portion proximal said surface, said first portion comprising (N)ₙ wherein N is a nucleotide and n is about 5 to about 50, and (ii) a second portion distal said surface, said second portion comprising a polynucleotide and
(b) a reagent reactive with said first portion.

36. A kit according to claim 35 wherein N is A.

37. A kit according to claim 35 wherein said support comprises at least one planar surface.

38. A kit according to claim 35 wherein said support comprises glass.
